(19) United States
(12) Patent Application Publication
(10) Pub. No.: US 2021/0103807 A1
(43) Pub. Date: Apr. 8, 2021

(54) COMPUTER IMPLEMENTED METHOD AND SYSTEM FOR RUNNING INFERENCE QUERIES WITH A GENERATIVE MODEL

(71) Applicant: Babylon Partners Limited, London (GB)

(72) Inventors: Adam BAKER, London (GB); Albert BUCHARD, London (GB); Konstantinos GOURGOULIAS, London (GB); Christopher HART, London (GB); Saurabh JOHRI, London (GB); Maria Dolores Lomeli GARCIA, London (GB); Christopher LUCAS, London (GB); Iurii PEROV, London (GB); Robert WALECKI, London (GB); Max ZWIESSELE, London (GB)

(21) Appl. No.: 16/594,957

(22) Filed: Oct. 7, 2019

(51) Int. Cl. G06N 3/08; G06N 3/04; G16H 50/20

(52) U.S. Cl. CPC G06N 3/08; G16H 50/20; G06N 3/04

(57) ABSTRACT

Methods for performing inference on a generative model are provided. In one aspect, a method includes receiving a generative model in a probabilistic program form defining variables and probabilistic relationships between variables, and producing a neural network to model the behaviour of the generative model. The input layer includes nodes corresponding to the variables of the generative model, and the output layer includes nodes corresponding to a parameter of the conditional marginal of the variables of the input layer. The method also includes training the neural network using samples from the probabilistic program. A loss function is provided for each node of the output layer. The loss function for each output node is independent of the loss functions for the other nodes of the output layer. The method also includes performing amortised inference on the generative model. Systems and machine-readable media are also provided.

### FIELD

Embodiments of the present invention relate to the field of computer implemented determination methods and systems.

### BACKGROUND

Probabilistic programming languages (PPL) are used to define probabilistic programs. PPLs are used to formalise knowledge about the world and for reasoning and decision-making. They have been successfully applied to problems in a wide range of real-life applications including information technology, engineering, systems biology and medicine, among others.

Probabilistic Graphical Models (PGMS) can be expressed as programs in a PPL, and they provide a natural framework for expressing the probabilistic relationships between random variables in numerous fields across the natural sciences. Bayesian networks, a directed form of graphical model, have been used extensively in medicine, to capture causal relationships between entities such as risk-factors, diseases and symptoms, and to facilitate medical decision-making tasks such as disease diagnosis. Key to decision-making is the process of performing probabilistic inference to update one's prior beliefs about the likelihood of a set of diseases, based on the observation of new evidence.

### BRIEF DESCRIPTION OF FIGURES

FIG. 1 is an overview of a system in accordance with an embodiment;

FIG. 2(a) is a schematic diagram of a simple graphical model and FIG. 2(b) is a schematic of the stages in a probabilistic programming settings;

FIG. 3 is a flow diagram describing how inference is performed in accordance with an embodiment;

FIGS. 4(a), (b) and (c) are schematics of examples of structures of generative models upon which inference can be performed;

FIG. 5 is a plot demonstrating sampling from a probabilistic program;

FIG. 6 is a schematic of an overview of the training of a system in accordance with an embodiment;

FIG. 7 is a flow diagram showing the training of an example of a discriminative model to use the method of FIG. 3;

FIG. 8 is a flow diagram showing the use of the trained model with the inference engine of FIG. 6;

FIG. 9 is a schematic of a system in accordance with an embodiment.

### DETAILED DESCRIPTION

In an embodiment, a probabilistic programming system is provided for performing inference on a generative model, the probabilistic programming system being adapted to: allow a generative model to be expressed, said generative model defining variables and probabilistic relationships between variables, wherein the variables comprise hidden and observed variables; condition values of unknown variables in the model using evidence, wherein said evidence populates observed variables; and perform amortised inference on said generative model, wherein the probabilistic program performs amortised inference by: acquiring a trained neural network, said neural network being trained neural network wherein said training was performed using samples derived from said probabilistic program and wherein the training was performed by masking some of the data of the samples, wherein the same trained model is acquired for a generative model regardless of the observed evidence; generating a data driven proposal from said trained neural network using said evidence; and using said data driven proposal as a proposal for amortised inference.

Generative models (presented as probabilistic graphical models) now form the backbone of many decision and diagnosis systems. Such models can be expressed in a probabilistic programming language (PPL) related systems that allows inference to be performed more easily. The disclosed systems and methods solve a technical problem with a technical solution, namely to provide faster inference for a probabilistic program by performing amortised inference. The amortised inference stage uses a discriminative model that has been trained by masking some of the variables. This means that the same neural network can provide a proposal for amortised inference regardless of the observed evidence. Thus, only a single trained discriminative model needs to be stored in memory to handle all evidence. This reduces the memory requirements of the system. The trained discriminative model thus can be incorporated as part of the amortised inference stage of a PPL and can be viewed as part of a compiler for the PPL.

During the inference stage, the PPL will generate samples to be produced by the sampling stage. Each sample can be viewed as a thread or run through the PGM where during the collection of each sample, variables are stored in memory or accumulated in aggregated statistics (e.g., mean or variance). By using a data driven proposal from the discriminative model, the number of samples required can be reduced and therefore the number of accesses within the memory and the number of calls to a processor to perform the sampling process are reduced. Further, the closer proposal distribution to the target distribution, the smaller the number of samples required.

In an embodiment, the discriminative model is trained such that it allows the prediction of both categorical and continuous variables for a range of PGMs with different graphical structures. The above therefore allows the system to produce answers using such new approximate inference with the accuracy comparable to using exact or already existing approximate inference techniques, but in a fraction of the time and with a reduction in the processing required. The inference engine may be configured to perform importance sampling over conditional marginal. However, other methods may be used such as Variational Inference, other Monte Carlo methods, etc.

The above embodiment will allow the performance of amortised inference on the generative model by providing any possible evidence (that matches this generative model) to the trained neural net and using the output of the trained neural net as a proposal distribution for the amortised inference over all other variables.

In a further embodiment, a method of performing inference on a generative model is provided, the method comprising: receiving a generative model in a probabilistic program form, said probabilistic program form defining variables and probabilistic relationships between variables; producing a neural network to model the behaviour of said generative model, wherein the input layer of said neural network comprises a plurality of nodes corresponding to the variables of said generative model and the output layer comprises a plurality of nodes corresponding to a parameter of the conditional marginal of the variables of the input layer; training the neural network using masked samples from said probabilistic program and wherein a loss function is provided for each node of the output layer, the loss function for each output node being independent of the loss functions for the other nodes of the output layer; performing amortised inference on the generative model by providing evidence to the trained neural net and using the output of the neural net to facilitate the inference.

The variables comprise hidden and observed variables, the evidence populating observed variables.

In some embodiments, there are a plurality of different types of variables and a loss function is selected for each output node dependent on the type of variable. For example, the different types of variables are selected from: continuous variables, binary variables and categorical variables. In an embodiment, categorical cross entropy loss is the loss function used for output nodes with categorical values and mean square loss for nodes with continuous values. However, other loss functions could be used.

In an embodiment, producing a neural network comprising selecting the number of hidden layers or the number of nodes in each hidden layer of the network dependent on the architecture of the generative model.

Selecting the number of hidden layers and selecting the number of nodes for a discriminative model may comprise: producing a plurality of training samples from the generative model using said probabilistic programming framework; producing a test discriminative network with N hidden layers and M hidden nodes per layer, where N and M are integers; training the test discriminative network to determine a measure of the loss; repeating the process for different values of M and N and selecting the discriminative network with the lowest loss function.

The values of M and N are determined using a randomised grid search and/or using two-fold cross validation.

In a further embodiment, a method of producing a neural network from a generative model is provided wherein said generative model is in a probabilistic program form, said probabilistic program form defining variables and probabilistic relationships between variables, the method comprising: producing a neural network to model the behaviour of said generative model, wherein the input layer of said neural network comprises a plurality of nodes corresponding to the variables of said generative model and the output layer comprises a plurality of nodes corresponding to a parameter of the conditional marginal of the variables of the input layer; selecting the number of hidden layers and hidden nodes for a discriminative model per layer using samples from said probabilistic program; and training the neural network using samples from said probabilistic program and wherein a loss function is provided for each node of the output layer, the loss function for each output node being independent of the loss functions for the other nodes of the output layer.

In one embodiment, the method relates to a medical inference method wherein the generative model describes the relationships between diseases and evidence.

In the above structure, diseases can be represented as both hidden and observed variables. This allows the effects of one or more diseases that the patient is known on a further disease to be modelled.

The generative model is not limited to a two or three layer PGM and may have a layer, chain, star, grid or any other structure.

In an embodiment, a method for providing computer implemented medical diagnosis is provided, the method comprising: receiving an input from a user comprising evidence of the user; providing the evidence as an input to a discriminative model that has been trained to output the conditional probability of the user having one or more diseases conditioned on the evidence, wherein the discriminative model has been pre-trained to approximate a probabilistic programming framework defining probabilistic relationships between observed and latent variables, wherein the variables are nodes, the variables comprising both categorical and continuous variables, wherein some of the latent variables correspond to diseases and the evidence corresponds to an observed variable; the discriminative model being trained using samples from said probabilistic programming framework, the training of the discriminative model using a first loss function at the output node for categorical variables and a second loss function at the output node for continuous variables, and outputting the conditional probability of the user having one or more diseases conditioned on the evidence.

In an embodiment, a system for performing inference on a generative model is provided, the system comprising: a processor and a memory, the processor being configured to: receive a generative model in a probabilistic program form, said probabilistic program form defining variables and probabilistic relationships between variables; produce a neural network to model the behaviour of said generative model, wherein the input layer of said neural network comprises a plurality of nodes corresponding to the variables of said generative model and the output layer comprises a plurality of nodes corresponding to a parameter of the conditional marginal of the variables of the input layer; train the neural network using samples from said probabilistic program and wherein a loss function is provided for each node of the output layer, the loss function for each output node being independent of the loss functions for the other nodes of the output layer; and perform amortised inference on the generative model by providing evidence to the trained neural net and using the output of the discriminative model for the amortised inference on the generative model.

In an embodiment, a system for providing computer implemented medical diagnosis is provided, the system comprising: a processor and a memory, the processor being adapted to: receive an input from a user comprising evidence of the user; retrieve from the memory a discriminative model that has been trained to output the conditional probability of the user having one or more diseases conditioned on the evidence; provide the evidence from the user as an input to the discriminative model; and output the conditional probability of the user having one or more diseases conditioned on the evidence. Wherein the discriminative model has been pre-trained to approximate a probabilistic programming framework defining probabilistic relationships between observed and latent variables, wherein the variables are nodes, the variables comprising both categorical and continuous variables, wherein some of the latent variables correspond to diseases and the evidence corresponds to an observed variable; the discriminative model being trained using samples from said probabilistic programming framework, the training of the discriminative model using a first loss function at the output node for categorical variables and a second loss function at the output node for continuous variables.

To give context to one possibly use of system in accordance with an embodiment, an example will be discussed in relation to the medical field. However, embodiments described herein can be applied to any inference problem on a generative model.

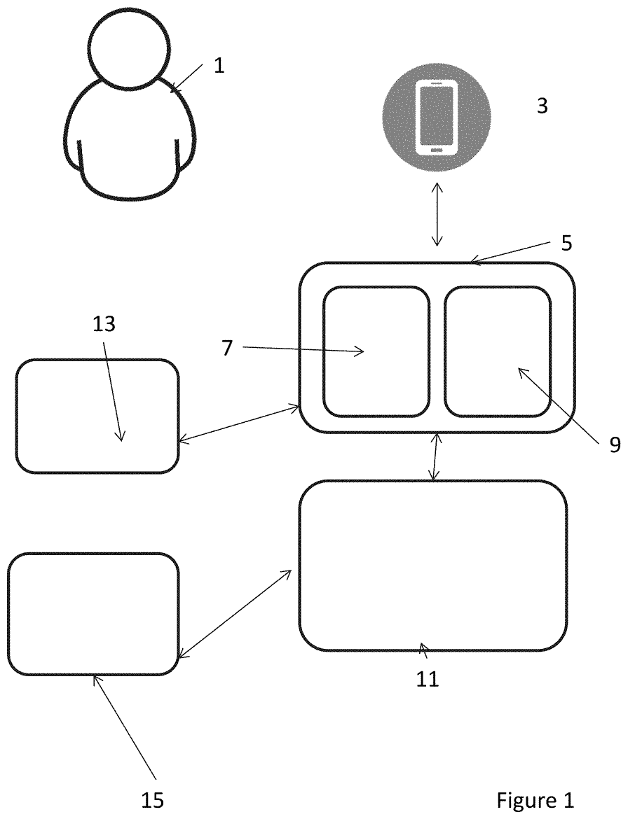

FIG. 1 is a schematic of a diagnostic system. In one embodiment, a user 1 communicates with the system via a mobile phone 3. However, any device could be used, which is capable of communicating information over a computer network, for example, a laptop, tablet computer, information point, fixed computer etc.

The mobile phone 3 will communicate with interface 5. Interface 5 has 2 primary functions, the first function 7 is to take the words uttered by the user and turn them into a form that can be understood by the inference engine 11. The second function 9 is to take the output of the inference engine 11 and to send this back to the user's mobile phone 3.

In some embodiments, Natural Language Processing (NLP) is used in the interface 5. NLP helps computers interpret, understand, and then use everyday human language and language patterns. It breaks both speech and text down into shorter components and interprets these more manageable blocks to understand what each individual component means and how it contributes to the overall meaning, linking the occurrence of medical terms to the Knowledge Graph. Through NLP it is possible to transcribe consultations, summarise clinical records and chat with users in a more natural, human way.

However, simply understanding how users express their symptoms and risk factors is not enough to identify and provide reasons about the underlying set of diseases. For this, the inference engine 11 is used. The inference engine is a powerful set of machine learning systems, capable of reasoning on a space of >100 s of billions of combinations of symptoms, diseases and risk factors, per second, to suggest possible underlying conditions.

In an embodiment, the Knowledge Graph 13 is a large structured medical knowledge base. It captures human knowledge on modem medicine encoded for machines. This is used to allow the above components to speak to each other. The Knowledge Graph keeps track of the meaning behind medical terminology across different medical systems and different languages.

In an embodiment, the patient data is stored using a so-called user graph 15.

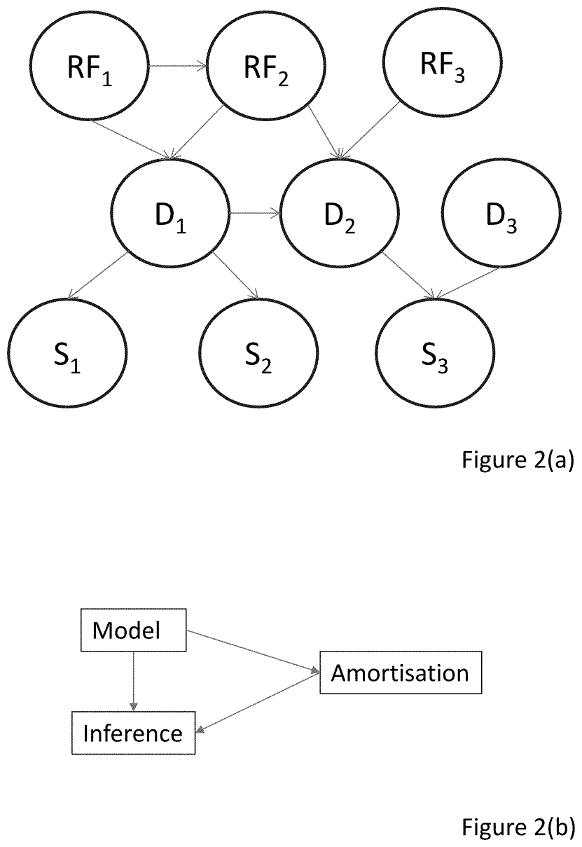

In an embodiment, the inference engine 11 comprises a generative model that may be a probabilistic graphical model or any type of probabilistic framework. FIG. 2 is a depiction of a probabilistic graphical model of the type that may be used in the inference engine 11 of FIG. 1.

In this specific embodiment, to aid understanding, a 3 layer Bayesian network will be described, where one layer related symptoms, another to diseases and a third layer to risk factors. However, the methods described herein can relate to any collection of variables where there are observed variables (evidence) and latent variables.

The graphical modeling is a natural framework for expressing probabilistic relationships between random variables, to facilitate causal modelling and decision making. In the model of FIG. 2, when applied to diagnosis, D stands for disease, S for symptom and RF for Risk Factor. Three layers: risk factors, diseases and symptoms. Risk factors causes (with some probability) influence other risk factors and diseases, diseases causes (again, with some probability) other diseases and symptoms. There are prior probabilities and conditional marginals that describe the “strength” (probability) of connections.

In this simplified specific example, the model is used in the field of diagnosis. In the first layer, there are three nodes S₁, S₂ and S₃, in the second layer there are three nodes D₁, D₂ and D₃ and in the third layer, there are three nodes RF₁, RF₂ and RF₃.

In the graphical model of FIG. 2, each arrow indicates a dependency. For example, D₁ depends on RF₁ and RF₂. D₂ depends on RF₂, RF₃ and D₁. Further relationships are possible. In the graphical model shown, each node is only dependent on a node or nodes from a different layer. However, nodes may be dependent on other nodes within the same layer.

The embodiments described herein relate to the inference engine.

In an embodiment, in use, a user 1 may input their symptoms via interface 5. The user may also input their risk factors, for example, whether they are a smoker, their weight etc. The interface may be adapted to ask the patient 1 specific questions. Alternately, the patient may just simply enter free text. The patient's risk factors may be derived from the patient's records held in a user graph 15. Therefore, once the patient identified themselves, data about the patient could be accessed via the system.

In further embodiments, follow-up questions may be asked by the interface 5. How this is achieved will be explained later. First, it will be assumed that the patient provide all possible information (evidence) to the system at the start of the process.

The evidence will be taken to be the presence or absence of all known symptoms and risk factors. For symptoms and risk factors where the patient has been unable to provide a response, these will assume to be unknown.

Next, this evidence is passed to the inference engine 11. In an embodiment, inference engine 11 performs Bayesian inference on PGM of FIG. 2(a). The PGM of FIG. 2(a) will be described in more detail with reference to FIG. 2(a) after the discussion of FIG. 1.

Due to the size of the PGM, it is not possible to perform exact inference in a realistic timescale. Therefore, the inference engine 11 performs approximate inference.

When performing approximate inference, the inference engine 11 requires an approximation of the conditioned probability distributions within the PGM to act as proposals for the sampling.

A PGM can be defined using a probabilistic programming language (PPL) in a probabilistic programming framework. In a probabilistic program nodes and edges are used to define a distribution p(x, y). Here, x are the latent variables and y are the observations.

The purpose of a probabilistic program is to implicitly specify a probabilistic generative model.

In an embodiment, probabilistic program systems will be considered to be systems such that: (1) the ability to define a probabilistic generative model in a form of a program, (2) the ability to condition values of unknown variables in a program such that this allows data from real world observations to be incorporated into a probabilistic program and infer the posterior distribution over those variables. In some probabilistic programs, this is achieved via observe statements.

FIG. 2(b) shows the basic building blocks of a probabilistic program: 1) Defining a Model; 2) Inference given Observations, and optionally 3) Amortisation

Probabilistic programs are capable of calling on a library of probabilistic distributions that allow variables to be generated from the distributions in a model definition step. Such distributions can be selected from, but not limited to Bernoulli; Gaussian; Categorical etc:

Examples of possible sampling steps are:

### Variable1=Bernoulli(μ)

### Variable2=Gaussian(μ,σ)

### Etc

In the above, Variable2 sampled from the Normal distribution and μ and σ are the mean and standard deviation respectively.

Probabilistic programs can be used to represent probabilistic graphical models (PGM) which use graphs to denote conditional dependencies between random variables. The probability distributions of a PGM can be encoded in a probabilistic program by, for example, by encoding each distribution from which values are to be drawn. Different values for the parameters of a distribution can be set dependent on the variable of an earlier distribution in the probabilistic program. Thus, it is possible to encode complex PGMs.

As noted above, a probabilistic program can also be used to condition values of the variables. This can be used to incorporate real world observations. For example, in some syntax the command “Observe” will allow the output to only consider variables that agree with some real world observation.

For example:
Observe (c=1)
Would block all runs (samples) where the variable

The inference stage allows an implicit representation of a posterior multi variable probability distribution to be defined. The inference stage may use an exact inference approach, for example, junction tree algorithm etc. Approximate inference is also possible using, for example, importance sampling.

In Importance sampling, a function f is considered for which its expectation, Ep[f] is to be estimated, under some probability distribution P. It is often the case that we can evaluate P up to a normalizing constant.

In Importance Sampling, expectation Ep[f] is estimated by introducing a distribution Q, known as the proposal distribution, which can both be sampled and evaluated. This gives:

$$\begin{matrix}{E}_{p} [ f ] = \int f ( x ) P ( x ) dx \\ = \int f ( x ) \frac{P ( x )}{Q ( x )} Q ( x ) dx \\ = {lim}_{n \to ∞} \frac{1}{n} {\sum }_{i = 1}^{n} f ( {x}_{i} ) {ω}_{i} ,\end{matrix} \qquad ( 3 )$$

Where xᵢ˜Q and where wᵢ=P (xᵢ)/Q(xᵢ) are the importance sampling weights. If P can only be evaluated up to a constant, the weights need to be normalized by their sum.

In other examples, the inference stage allows the most likely values of the variables to be defined. In other embodiments, a probabilistic program allows for samples to be drawn, for example, to allow the test of a further model.

As noted above, there can also be an amortization stage. In one embodiment, the amortization stage uses a neural net trained on samples produced using samples from the prior of the model. The neural network that will be described in more detail with reference to FIG. 6, is trained using masking.

The trained neural network can then be used to produce a data driven proposal for the inference stage. For example, the trained neural network can be used to determine a proposal distribution as described above for importance sampling. The use of a data driven proposal reduces the computation required to be able to perform inference.

When doing approximate inference for a probabilistic program, the inference stage would often require many samples to be produced by the sampling stage. Each sample can be viewed as a thread or run through the PGM where during the collection of each sample, variables are stored in memory or accumulated in aggregated statistics (e.g., mean or variance). By using a data driven proposal, the number of samples required can be reduced and therefore the number of accesses within the memory and the number of calls to a processor to perform the sampling process are reduced. Further, the closer the proposal distribution to the target distribution, the smaller generally the number of samples required.

As explained above, the neural net is trained using masking. This means that the neural net is trained to be robust to the observation and non-observation of various variables.

This in turn allows the same neural networks to be used regardless of which variables have been observed. This allows a single trained neural network to be used and continually called by the same probability programming language regardless of the status of the observed variables. This has significant advantages in terms of making the inference more efficient by amortising it, as well as by using just one network for all possible combinations of observed/unobserved nodes, hence in terms of the memory footprint of the system.

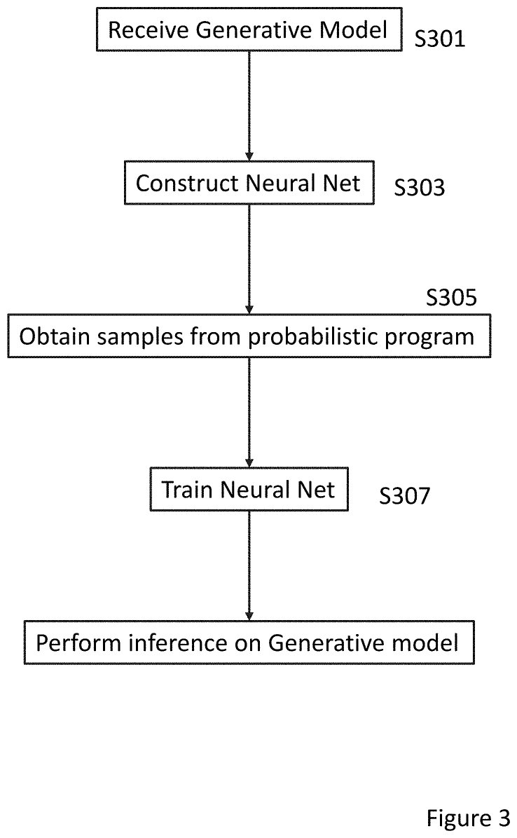

FIG. 3 sets out an inference method that can be used in the inference stage of the probabilistic program. In FIG. 3, the inference method learns from the prior samples from a generative model written as a probabilistic program with a bounded number of random choices without any separation into hidden and observed variables beforehand. The process of learning happens into a discriminative model that is later used for amortised inference posterior with any chosen set of observed variables for hidden variable conditional marginals.

The generative model which is received in step S301 can be the above PGM or another probabilistic model expressed as a probabilistic program.

In step S303, a neural net is then constructed as a discriminative model. In an embodiment, the input layer of the neural network will have a plurality of nodes, each corresponding to a variable (both hidden and observed) of the probabilistic programming model. The output layer of the neural network also has a node corresponding to each of the variables of the input layer. However, the nodes of the output layer each express a conditional marginal probability of the variable having a predefined value conditioned on the observed evidence. For example, if the variable is a binary variable, the variable could take a value of true or false. In this situation, the conditional marginal probability of the output node is that the variable is true conditioned on the observed variables.

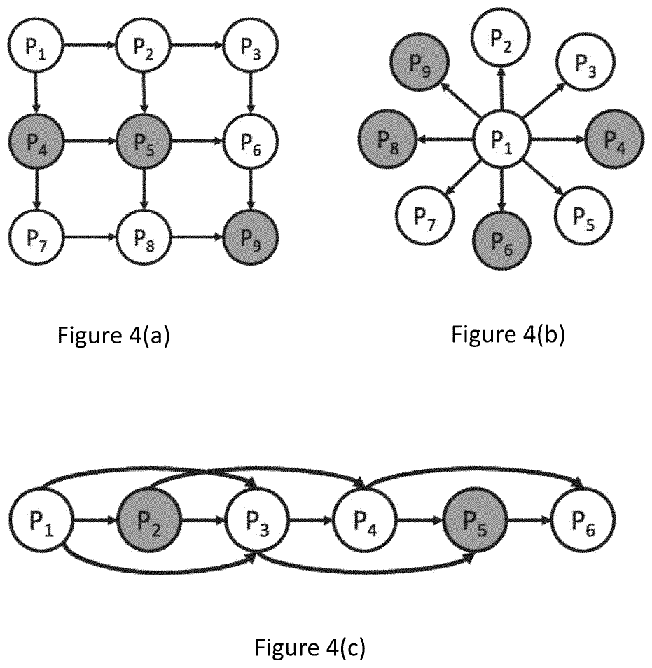

In addition to the number of input nodes and output nodes, the number of hidden layers within the network and the number of hidden nodes for each layer can be selected. In an embodiment, these two hyper parameters can be selected based on the design of the generative model. FIGS. 4(a) to (c) show different structures for the generative model. For example, more hidden layers may be applied for a more complex generative model. In an embodiment, two-fold cross validation is applied to select the best parameters. For this, a test set was created with marginals (using the synthetic graphs) and then evaluated the model with different parameters. A randomized grid search was used to find the best parameters

Once the neural network is trained it can be stored in memory and retrieved each time, there is no need to retrain or generate the neural network each time.

The discriminative model or neural network is then trained to approximate any possible posterior P(X|Y) with any possible X and Y such that X∪Y=Z. In an embodiment, this is achieved using an amortised inference-based method for efficient computation of conditional posterior probabilities in probabilistic programs. This trained discriminative model will be termed the Universal Marginaliser (UM). In general, the UM is not restricted to particular types of observations or probabilistic programs.

In an embodiment, the Universal Marginaliser (UM) is based on a feed-forward neural network, used to perform fast, single-pass approximate inference on probabilistic programs at any scale. In this section we introduce the notation and discuss the UM building and training algorithm.

A probabilistic program can be defined by a probability distribution P over sequences of executions on random variables Z={X₁, . . . X_{N}}. For each inference request, the random variables are divided into two disjoint sets, Y⊂Z the set of observations within the program, and X⊂Z\Y the set of latent nodes; note that the same UM can deal with any possible combination of these two sets.

In an embodiment, a neural network is utilised to learn an approximation to the values of the conditional posterior marginal distribution for each variable Xᵢ∈X given an instantiation Y of observations. For a set of variables Xᵢ with i∈1, . . . N, the desired neural network maps the vectorised representation of Y to the approximations of a conditional marginal distributions. This NN is used as a function approximator, and hence can approximate any posterior marginal distribution given an arbitrary set of observations Y. For this reason, such discriminative model is called the Universal Marginaliser (UM).

Once the weights of the NN are optimised, it can be used as an approximation for the conditional marginals of hidden variables X given the observations Y. It also can be used to compute the hidden variable proposal for each Xᵢ sequentially given all previous X₁ . . . Xᵢ₋₁ and observations (i.e. using ancestral sampling).

In an embodiment, the UM is trained with a minimum effort of hyperparameter tuning. To this end, the neural network architecture of the UM is specific for the type of the target probabilistic program and is automatically selected based on predefined rules. On the output layer for example, a categorical cross-entropy loss is deployed for nodes with categorical states and mean square error loss for nodes with continuous values. Furthermore, in an embodiment, an ADAM optimization method with an initial learning rate of 0.001 and a learning rate decay of 10⁻⁴ is used for each of the losses. The two model parameters to be set by the user or found by hyperparameter optimization are h, the number of hidden layers and s, the number of hidden nodes per layer. In an embodiment a deeper and more complex network can be used for larger probabilistic programs. However, embodiments show that even shallow and simple networks are capable of learning complex dependencies. In an embodiment, the UM framework is implemented in the Pyro PPL and the deep learning platform PyTorch.

In practice, optimisation is applied on batches rather than on a full training set, and batches are directly sampled from the probabilistic program. This improves memory efficiency during training and ensures that the network receives a large variety of observations, accounting for low probability regions in P.

To train the UM, samples are obtained from the probabilistic program in step S305.

An example of a probabilistic program is shown below:

```
def probProg (t1, v);
  for i in [2, 3, ....... ,50]:
    if abs(t[i−1])<1
    t[i] − Bernoulli (abs(t[i−1]))
    else:
    t[i] − Gaussian (t[i−1],v)
  return
```

In this simplified program, binary values are sampled from the Bernoulli distribution and floats from the Gaussian distribution. ‘t1’ is just the initial value and in t2 will be either a binary value or float, depending if Bernoulli (abs(t1)) is true or false. The standard deviation of the Gaussian is denoted with ‘v’. In this simplified program, the value of t2 is generated from the value of t1, then the value of t3 is generated from the value of t2 and so on. The output in this probabilistic program is [t1, t2, t3, . . . , t50]. Each of t1, . . . t50 can, for example, be considered to be a node in a PGM.

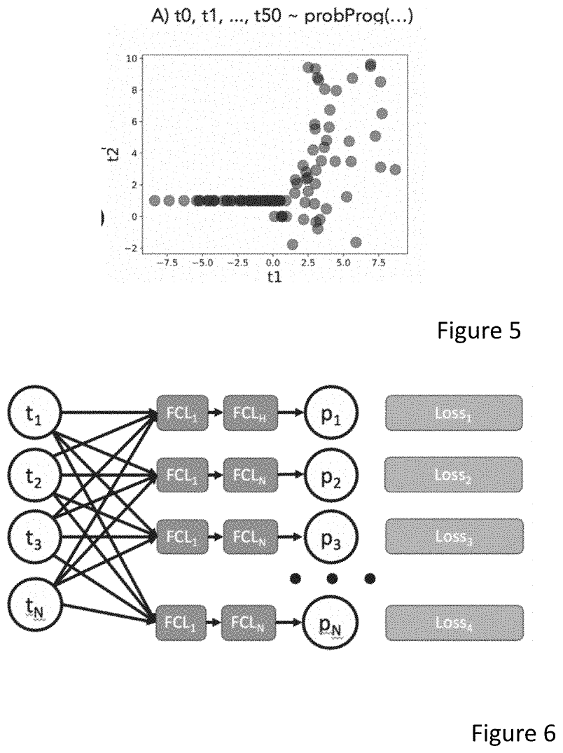

FIG. 5 shows an example output from the above program, where different samples for the value of t2 dependent on t1 are shown.

It should be noted that during runtime it is not known if each output (t_i) is binary or a float. Therefore, it is very difficult to build a general purpose UM for such a program. In an embodiment, for each random variable in a probabilistic program there might be as a many variables in a discriminative model as there are different types that the random variable can take (e.g., binary type and float type). In an embodiment, this problem is also addressed by selecting different loss functions for the different types of outputs.

The nodes of the PGM, in this example, t1 to t50 are then to be used as the input layer to UM shown in FIG. 6. In FIG. 6, FCL is used to denote a fully connected layer.

For each iteration, a batch of observations from the program is sampled and used for training.

To train the UM, the samples are then masked. In order for the network to approximate the marginal posteriors at test time, and be able to do so for any input observations, each sample Si is prepared by masking. The network will receive as input a vector where a subset of the nodes initially observed were replaced by the priors or special constant distinguishable values. This augmentation can be deterministic, i.e., always replace specific nodes, or probabilistic. In an embodiment, a constantly changing probabilistic method is used for masking. This is achieved by randomly masking i nodes where i is a random number, sampled from a uniform distribution between 0 and N. This number changes with every iteration and so does the total number of masked nodes.

Finally, the NN is trained by minimising multiple losses, where each loss is specifically designed for each of the random variables in the probabilistic program. In an embodiment, categorical cross-entropy loss is used for categorical values and mean square error for nodes with continuous values. In an embodiment, a different optimiser is used for each output and minimise the losses independently. This ensures that the global learning rates are also updated specifically for all random variables.

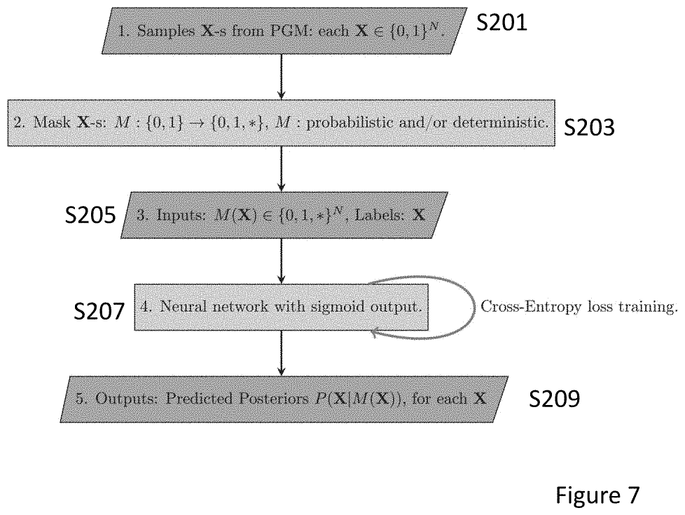

The training of the UM will be described in detail with reference to FIG. 7. However, the UM is a model that can approximate the behaviour of the entire PGM. In one embodiment the UM is a single neural net, in another embodiment, the model is a neural network which consists of several sub-networks, such that the whole architecture is a form of auto-encoder-like model but with multiple branches. Further, the UM as will be described with reference to FIG. 7 is trained to be robust to the user giving incomplete answers. This is achieved via the masking procedure for training the UM that was mentioned above and will now be described with reference to FIG. 7.

The training process for the above described UM involves generating samples from the probabilistic program, in each sample masking some of the nodes, and then training with the aim to learn a distribution over this data. This process is explained through the rest of the section and illustrated in FIG. 7.

The UM can be trained off-line using the samples generated in S303 of FIG. 3. In an embodiment these are unbiased samples that are generated from the probabilistic graphical model (PGM) using ancestral sampling. Each sample is a vector that will be the values for the classifier to learn to predict.

In an embodiment, for the purpose of prediction, some nodes in the sample then be hidden, or “masked” in step S203. This masking is either deterministic (in the sense of always masking certain nodes) or probabilistic over nodes. In embodiment each node is probabilistically masked (in an unbiased way), for each sample, by choosing a masking probability P˜U[0,1] and then masking all data in that sample with probability p.

The nodes which are masked (or unobserved when it comes to inference time) are represented consistently in the input tensor in step S205.

The neural network is then trained using multiple loss functions, one loss function for each output.

In a further embodiment, the output of the neural net can be mapped to posterior probability estimates. However, when e.g., the cross entropy loss is used for binary variables, the output from the neural net is exactly the predicted probability distribution.

The trained neural network can then be used to obtain the desired probability estimates by directly taking the output of the sigmoid layer. This result could be used as a posterior estimate. It also can be used for performing amortised inference.

Thus a discriminative model is now produced which, given any set of observations xₒ, will approximate all the posterior marginals in step S209. Note that the training of a discriminative model can be performed, as often practised, in batches; for each batch, new samples from the model can be sampled, masked and fed to the discriminative model training algorithm; all sampling, masking, and training can be performed on Graphics Processing Units.

FIG. 6 shows a schematic of a possible neural network. The input nodes, T1, T2 etc. correspond to the variables of the generative model. The hidden layers, FCL one etc. output to the output nodes that indicate the probability distribution. In an embodiment, this will be the mean and variance.

Shown in FIG. 6, each output has its own separate loss.

The formation of the loss function is dependent on the nature of the variable as discussed above.

By designing the neural network in the ways described above, it is possible to handle both binary, categorical and continuous variables within the same network. It is also possible to model the effect of 2 or more diseases within the network.

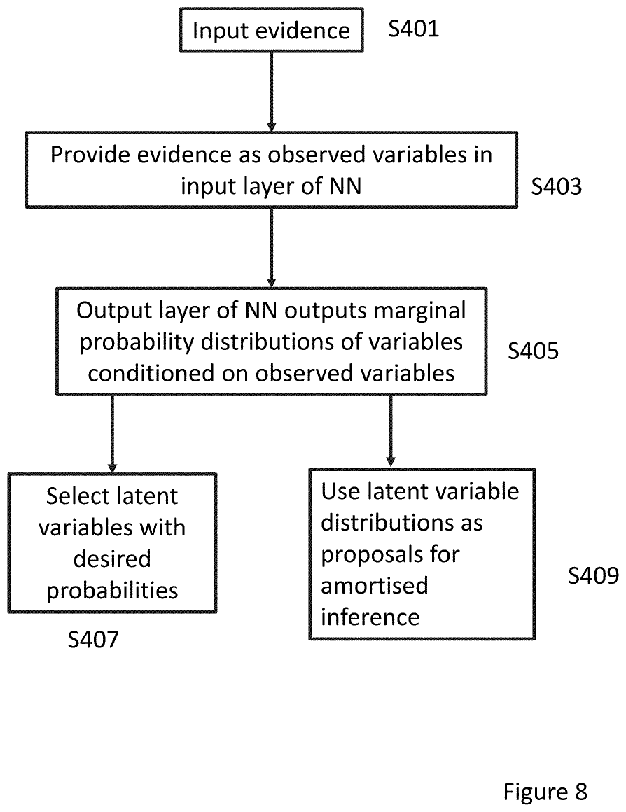

FIG. 8 shows a flowchart indicating how inference is performed using the trained neural network. First, evidence is input in step S401. For example, with a medical diagnosis network, the evidence might be the symptoms of the user, risk factors all pre-existing unknown diseases.

These are then provided the input layer of the NN as observed variables in step S403.

The output layer of the neural net then outputs in step S405 the parameters that define the distribution of the marginal probability distribution for that variable conditioned on the observable variables or evidence.

In step S407, depending on the question asked of the generative model, an answer can be given. For example, if the generative model relates to medical diagnosis, the nodes of the output layer that relate to diseases or potential causes for the symptoms can be compared and those nodes which show a more likely disease can be considered to be the answer. Where there are a number of possible diseases that caused the symptoms, the NN can be used again to determine the evidence that would be needed to further reduce the number of possible diseases.

As an alternative, to step S407, the produced latent variable distributions can then be used as proposals for amortised inference in step S409.

It is possible using a value of information analysis (VoI) to determine from the above distributions whether asking a further question would improve the probability of diagnosis. For example, if the initial output of the system seems that there are 9 diseases each having a 10% likelihood based on the evidence, then asking a further question will allow a more precise and useful diagnosis to be made. In an embodiment, the next further questions to be asked are determined on the basis of questions that reduce the entropy of the system most effectively.

In one embodiment, the analysis to determine whether a further question should be asked and what that question should be is based purely on the output of the UM that provide an estimate of the probabilities.

Once the user supplies further information, then this is then passed back and forth to the inference engine 11 to update evidence to produce updated probabilities.

To demonstrate the above two types of training methods were compared with three different network architectures and eight different probabilistic programs (see FIGS. 4(a) to (c)). The first method serves as a baseline. It is a neural network, where the losses of all outputs are summed and jointly minimised.

We refer to this method as NNs, where s indicates the size of the network. For the second method, different optimisers and different losses are used for each output. This will be referred to as UMs. The architectures of UM1/NN1 are identical. The networks have 2 hidden layers with 10 nodes each. UM2/NN2 have 4 hidden layers with 35 nodes each and UM3/NN3 have 8 hidden layers with 100 nodes. The quality of the predicted posteriors was measured using a test set computed for 100 sets of observations via importance sampling with one million samples. Table 1 shows the performance in terms of correlation of various neural networks for marginalisation.

| | Chain | Chain | Chain | Grid | Grid | Star | Star | Star |
|---|---|---|---|---|---|---|---|---|
| | 4 | 16 | 32 | 9 | 16 | 4 | 8 | 32 |
| NN₁ | 0.903 | 0.875 | 0.698 | 0.877 | 0.926 | 0.914 | 0.822 | 0.667 |
| NN₂ | 0.932 | 0.852 | 0.795 | 0.824 | 0.904 | 0.920 | 0.821 | 0.804 |
| NN₃ | 0.927 | 0.837 | 0.631 | 0.843 | 0.919 | 0.900 | 0.756 | 0.783 |
| UM₁ | 0.945 | 0.859 | 0.703 | 0.875 | 0.928 | 0.919 | 0.907 | 0.697 |
| UM₂ | 0.935 | 0.890 | 0.823 | 0.889 | 0.958 | 0.919 | 0.908 | 0.811 |
| UM₃ | 0.913 | 0.846 | 0.609 | 0.923 | 0.922 | 0.933 | 0.882 | 0.789 |

The UM can be used either directly as an approximation of probabilities or it can be used as a proposal for amortised inference. The above embodiments propose an idea of automatic generation and training of a neural network given a probabilistic program and samples from its prior, such that later that neural network can be used as a proposal for performing the posterior inference given any possible evidence set. Such framework could be implemented in one of probabilistic programming platforms, e.g., in Pyro. While this approach directly could be applied only to the models with bounded number of random choices, it might be possible to map the “names” of random choices in a program with finite but unbounded number of those random choices to the bounded number of names using some schedule, hence performing a version of approximate inference in sequence.

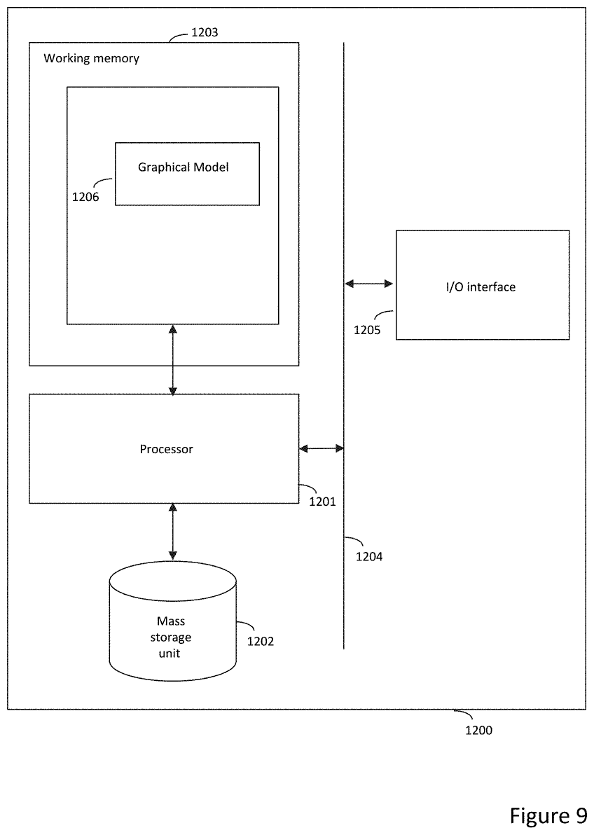

While it will be appreciated that the above embodiments are applicable to any computing system, an example computing system is illustrated in FIG. 9, which provides means capable of putting an embodiment, as described herein, into effect. As illustrated, the computing system 1200 comprises a processor 1201 coupled to a mass storage unit 1202 and accessing a working memory 1203. As illustrated, a graphical model 1206 is represented as software products stored in working memory 1203. However, it will be appreciated that elements of the graphical model 1206 described previously, may, for convenience, be stored in the mass storage unit 1202.

Depending on the use, the graphical model 1206 may be used with a chatbot, to provide a response to a user question.

Usual procedures for the loading of software into memory and the storage of data in the mass storage unit 1202 apply. The processor 1201 also accesses, via bus 1204, an input/output interface 1205 that is configured to receive data from and output data to an external system (e.g., an external network or a user input or output device). The input/output interface 1205 may be a single component or may be divided into a separate input interface and a separate output interface.

Thus, execution of the inference method by the processor 1201 will cause embodiments as described herein to be implemented.

The UM 1206 can be embedded in original equipment, or can be provided, as a whole or in part, after manufacture. For instance, UM 1206 can be introduced, as a whole, as a computer program product, which may be in the form of a download, or to be introduced via a computer program storage medium, such as an optical disk. Alternatively, modifications to existing causal discovery model software can be made by an update, or plug-in, to provide features of the above described embodiment.

The computing system 1200 may be an end-user system that receives inputs from a user (e.g., via a keyboard) and retrieves a response to a query using the UM 1206 adapted to produce the user query in a suitable form. Alternatively, the system may be a server that receives input over a network and determines a response. Either way, the use of the UM 1206 may be used to determine appropriate responses to user queries, as discussed with regard to FIG. 3 and FIG. 8.

Implementations of the subject matter and the operations described in this specification can be realized in digital electronic circuitry, or in computer software, firmware, or hardware, including the structures disclosed in this specification and their structural equivalents, or in combinations of one or more of them. Implementations of the subject matter described in this specification can be realized using one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

While certain embodiments have been described, these embodiments have been presented by way of example only, and are not intended to limit the scope of the inventions. Indeed the novel methods and systems described herein may be embodied in a variety of other forms; furthermore, various omissions, substitutions and changes in the form of methods and systems described herein may be made without departing from the spirit of the inventions. The accompanying claims and their equivalents are intended to cover such forms of modifications as would fall within the scope and spirit of the inventions.

1. A probabilistic programming system for performing inference on a generative model, the probabilistic programming system being adapted to:
allow a generative model to be expressed, said generative model defining variables and probabilistic relationships between variables, wherein the variables comprise hidden and observed variables;
condition values of unknown variables in the model using evidence, wherein said evidence populates observed variables; and
perform amortised inference on said generative model,
wherein the probabilistic program performs amortised inference by:
acquiring a trained neural network, said neural network being trained neural network wherein said training was performed using samples derived from said probabilistic program and wherein the training was performed by masking some of the data of the samples, wherein the same trained model is acquired for a generative model regardless of the observed evidence;
generating a data driven proposal from said trained neural network using said evidence; and
using said data driven proposal as a proposal for amortised inference.

2. A probabilistic programming system according to claim 1, wherein the variables are different types of variables are selected from: continuous variables; binary variables; and categorical variables.

3. A probabilistic programming system according to claim 1, wherein acquiring a trained neural network comprises:
producing a neural network to model the behaviour of said generative model, wherein the input layer of said neural network comprises a plurality of nodes corresponding to the variables of said generative model and the output layer comprises a plurality of nodes corresponding to a parameter of the conditional marginal of the variables of the input layer;
training the neural network using samples from said probabilistic program and wherein a loss function is provided for each node of the output layer, the loss function for each output node being independent of the loss functions for the other nodes of the output layer.

4. A probabilistic programming system according to claim 3, wherein there are a plurality of different types of variables and loss function is selected for each output node dependent on the type of variable.

5. A probabilistic programming system according to claim 4, wherein categorical cross entropy loss is the loss function used for output nodes with categorical values and mean square loss for nodes with continuous values.

6. A probabilistic programming system according to claim 3, wherein producing a neural network comprising selecting the number of hidden layers of the network dependent on the architecture of the generative model.

7. A probabilistic programming system according to claim 3, wherein producing a neural network comprising selecting the number of nodes in each hidden layer of the network dependent on the architecture of the generative model.

8. A probabilistic programming system according to claim 3, wherein producing a neural network comprises selecting the number of hidden layers and selecting the number of nodes in each hidden layer of the network dependent on the architecture of the generative model.

9. A probabilistic programming system according to claim 8, wherein selecting the number of hidden layers and selecting the number of nodes comprises:
producing a plurality of training samples from the generative model using said probabilistic programming framework;
producing a test discriminative network with N hidden layers and M hidden nodes per layer, where N and M are integers;
training the test discriminative network to determine a measure of the loss;
repeating the process for different values of M and N and selecting the discriminative network with the lowest loss function.

10. A probabilistic programming system according to claim 9, wherein the values of M and N are determined using a randomised grid search.

11. A probabilistic programming system according to claim 9, wherein M and N are determined using two-fold cross validation.

12. A probabilistic programming system according to claim 1, wherein the generative model describes the relationships between diseases and evidence.

13. A probabilistic programming system according to claim 12, wherein diseases are represented as both hidden and observed variables.

14. A probabilistic programming system according to claim 1, wherein the generative model has a layer, chain, star or grid structure.

15. A method for providing computer implemented medical diagnosis, the method comprising:
receiving an input from a user comprising evidence of the user;
providing the evidence as an input to a discriminative model that has been trained to output the conditional probability of the user having one or more diseases conditioned on the evidence,
wherein the discriminative model has been pre-trained to approximate a probabilistic programming model defining probabilistic relationships between observed and latent variables, wherein the variables are nodes, the variables comprising both categorical and continuous variables, wherein some of the latent variables correspond to diseases and the evidence corresponds to an observed variable;
the discriminative model being trained using samples from said probabilistic programming model, the training of the discriminative model using a first loss function at the output node for categorical variables and a second loss function at the output node for continuous variables, and
outputting the conditional probability of the user having one or more diseases conditioned on the evidence.

16. A system for performing inference on a generative model, the system comprising:
a processor and a memory, the processor being configured to:
receive a generative model in a probabilistic program form, said probabilistic program form defining variables and probabilistic relationships between variables;
produce a neural network to model the behaviour of said generative model, wherein the input layer of said neural network comprises a plurality of nodes corresponding to the variables of said generative model and the output layer comprises a plurality of nodes corresponding to a parameter of the conditional marginal of the variables of the input layer;
train the neural network using samples from said probabilistic program and wherein a loss function is provided for each node of the output layer, the loss function for each output node being independent of the loss functions for the other nodes of the output layer; and
perform amortised inference on the generative model by providing evidence to the trained neural net and using the output of the trained neural net as a proposal distribution for the amortised inference.

17. A system for providing computer implemented medical diagnosis, the system comprising:
a processor and a memory, the processor being adapted to:
receive an input from a user comprising evidence of the user;
retrieve from the memory a discriminative model that has been trained to output the conditional probability of the user having one or more diseases conditioned on the evidence;
provide the evidence from the user as an input to the discriminative model;
output the conditional probability of the user having one or more diseases conditioned on the evidence,
wherein the discriminative model has been pre-trained to approximate a probabilistic programming model defining probabilistic relationships between observed and latent variables, wherein the variables are nodes, the variables comprising both categorical and continuous variables, wherein some of the latent variables correspond to diseases and the evidence corresponds to an observed variable, the discriminative model being trained using samples from said probabilistic programming framework, the training of the discriminative model using a first loss function at the output node for categorical variables and a second loss function at the output node for continuous variables; and
use the output of the trained neural net as a proposal distribution for the amortised inference for the generative model.

18. A probabilistic programming method for performing inference on a generative model, the method comprising:
expressing a generative model in a probabilistic program, said generative model defining variables and probabilistic relationships between variables, wherein the variables comprise hidden and observed variables;
conditioning values of unknown variables in the model using evidence, wherein said evidence populates observed variables; and
performing amortised inference on said generative model,
wherein the probabilistic program performs amortised inference by:
acquiring a trained neural network, said neural network being trained neural network wherein said training was performed using samples derived from said probabilistic program and wherein the training was performed by masking some of the data of the samples, wherein the same trained model is acquired for a generative model regardless of the observed evidence;
generating a data driven proposal from said trained neural network using said evidence; and
using said data driven proposal as a proposal for amortised inference.

19. A non-transitory machine-readable storage medium comprising machine-readable instructions for causing a processor to execute a method for performing inference on a generative model, the method comprising:
expressing a generative model in a probabilistic program, said generative model defining variables and probabilistic relationships between variables, wherein the variables comprise hidden and observed variables;
conditioning values of unknown variables in the model using evidence, wherein said evidence populates observed variables; and
performing amortised inference on said generative model,
wherein the probabilistic program performs amortised inference by:
acquiring a trained neural network, said neural network being trained neural network wherein said training was performed using samples derived from said probabilistic program and wherein the training was performed by masking some of the data of the samples, wherein the same trained model is acquired for a generative model regardless of the observed evidence;
generating a data driven proposal from said trained neural network using said evidence; and
using said data driven proposal as a proposal for amortised inference.

20. A non-transitory machine-readable storage medium comprising machine-readable instructions for causing a processor to execute a method for providing computer implemented medical diagnosis, the method comprising:
receiving an input from a user comprising evidence of the user;
providing the evidence as an input to a discriminative model that has been trained to output the conditional probability of the user having one or more diseases conditioned on the evidence,
wherein the discriminative model has been pre-trained to approximate a probabilistic programming model defining probabilistic relationships between observed and latent variables, wherein the variables are nodes, the variables comprising both categorical and continuous variables, wherein some of the latent variables correspond to diseases and the evidence corresponds to an observed variable;
the discriminative model being trained using samples from said probabilistic programming model, the training of the discriminative model using a first loss function at the output node for categorical variables and a second loss function at the output node for continuous variables, and
outputting the conditional probability of the user having one or more diseases conditioned on the evidence.
